(19) United States
(12) Patent Application Publication
(10) Pub. No.: US 2013/0103119 A1
(43) Pub. Date: Apr. 25, 2013

(54) HIGH-FREQUENCY TREATMENT DEVICE

(71) Applicants: Kazunori Yamanaka, Tokyo (JP); Iwao Yamazaki, Tokyo (JP)

(72) Inventors: Kazunori Yamanaka, Tokyo (JP); Iwao Yamazaki, Tokyo (JP)

(73) Assignee: YA-MAN LTD., Tokyo (JP)

(21) Appl. No.: 13/806,458

(22) PCT Filed: Jun. 17, 2011
(86) PCT No.: PCT/JP2011/063902

§ 371 Date: Dec. 21, 2012

(30) Foreign Application Priority Data
Jun. 21, 2010 (JP) 2010-140046

(51) Int. Cl. A61N 1/36

(52) U.S. Cl. CPC A61N 1/36014
USPC 607/62

(57) ABSTRACT

Disclosed is a high-frequency treatment device provided with: an oscillator circuit and an amplification circuit that generate a high-frequency signal; and a head that holds terminals (41x and 41y) that run the high-frequency signal generated by the oscillator circuit and the amplification circuit through skin. To address the problem that running an excessively strong high-frequency signal through skin carries the danger of causing burns or other accidents, the disclosed high-frequency treatment device is provided with a current-carrying region change means that changes the region of the skin to which current is applied, from one of the terminals to the other, while the high-frequency signal is being applied.

### TECHNICAL FIELD

The present invention relates to the technical field of a high-frequency treatment device which treats the skin by applying a high-frequency signal to the skin.

### BACKGROUND ART

A high-frequency treatment device applies a high-frequency signal to facial skin, skin of the hands and feet or the like so as to satisfy the requirements for anti-aging, acne cure, treatment of spots, freckles or other skin problems in order to produce a more beautiful skin. This kind of high-frequency treatment device is disclosed for example in Patent document 1.

With the conventional high-frequency treatment device, when a current-carrying stimulation mode is set by a user with an input operation, a prescribed electric pressure is applied between electrodes for skin-contact. Accordingly, a weak electric current is supplied between the electrodes which contact the user's skin. The current-carrying stimulation enables one to provide the aforementioned efficacy for producing beautiful skin.

### PRIOR ART DOCUMENT

### Patent Document

Patent Document 1:
International Publication No. WO2006/131997

### SUMMARY OF THE INVENTION

Electrodes of a conventional high-frequency treatment device are positionally-fixed on two points. Accordingly, it carries the danger of causing burns or other accidents, when the high-frequency signal is applied to the skin excessively (for example, when the high-frequency signal is applied more than 0.1 to 0.3 W/cm² in cycles of 4 to 10 seconds).

In order to solve the problems described above, the present invention allows one to inhibit the excessive high-frequency signal application to the skin so that burns or other accidents can be prevented from occurring.

The high-frequency treatment device according to the present invention is comprised of some means as follows: a signal generation means which generates a high-frequency signal; a pair of terminal means which transmits the high-frequency signal generated by said signal generation means to skin; a motion sensing means which senses movements of said pair of terminal means onto the skin; and a control means which controls the signal generation means such that while movements of at least one of the pair of terminal means is being sensed by the motion sensing means, it controls the signal generation means so as to increase the level of the high-frequency signal above a specified threshold value, whereas when movements of the pair of terminal means have become undetectable by the motion sensing means, it controls the signal generation means so as to decrease the level of the high-frequency signal below the specified threshold value.

The high-frequency treatment device according to the present invention further comprises a current-carrying condition measurement means which measures the current-carrying condition of the high-frequency signal, in addition to the respective aforementioned means. When movements of the pair of terminal means have become undetectable by the motion sensing means, the control means measures the current-carrying condition by means of the current-carrying condition measurement means. Subsequently, when the current-carrying condition reaches the predetermined state, it controls the signal generation means so as to decrease the level of the high-frequency signal below the specified threshold value.

### Advantageous Effects of Invention

As described above, according to the present invention, the signal application control means is provided with some means as follows: the motion sensing means which senses movements of the pair of terminal means onto skin; and the control means which controls the signal generation means such that while movements of at least one of the pair of terminal means are being sensed, the level of the high-frequency signal is increased above the specified threshold value, whereas when movements of the pair of terminal means have become undetectable, the high-frequency signal is decreased below the specified threshold value. Accordingly, it becomes possible to inhibit the excessive current application to the skin so that burns or other accidents can be prevented.

In addition, since the high-frequency signal is not stopped even if movements of the pair of terminal means have become undetectable, it becomes possible to continue applying the treatment in the safe condition wherein burns or other accidents are prevented.

The signal application control means according to the present invention is further comprised of the current-carrying condition measurement means which measures the current-carrying condition of the high-frequency signal. The control means measures the current-carrying condition by means of the current-carrying condition measurement means when movements of the pair of terminal means have become undetectable by the motion sensing means, and when the current-carrying condition reaches the predetermined condition, it controls the signal generation means so that the level of the high-frequency signal is decreased below the specified threshold value. Accordingly, the high-frequency signal application is kept being carried out from the point at which the pair of terminal means has stayed in the same position on the skin S until the current-carrying condition reaches the predetermined condition, and thereafter it is stopped. Therefore, the excessive current application to the skin S is inhibited so that burns or other accidents can be prevented.

### BRIEF DESCRIPTION OF THE DRAWINGS

FIG. 1 is a configuration diagram of the high-frequency treatment device according to the first reference example.

FIG. 2 is a flow chart which illustrates the operation of the high-frequency treatment device according to the first reference example.

FIG. 3 is a configuration diagram of the high-frequency treatment device according to the first reference example.

FIG. 4 is a flow chart which illustrates the operation of the high-frequency treatment device according to the first reference example.

FIG. 5 is a configuration diagram which illustrates variations of a head of the high-frequency treatment device according to the first reference example.

FIG. 6 is a configuration diagram of the high-frequency treatment device according to the second reference example.

FIG. 7 is a flow chart which illustrates the operation of the high-frequency treatment device according to the second reference example.

FIG. 8 is a configuration diagram of the high-frequency treatment device according to the second reference example.

FIG. 9 is a configuration diagram of the high-frequency treatment device according to the third reference example.

FIG. 10 is a flow chart which illustrates the operation of the high-frequency treatment device according to the third reference example.

FIG. 11 is a configuration diagram of the high-frequency treatment device according to the third reference example.

FIG. 12 is a flow chart which illustrates the operation of the high-frequency treatment device the third reference example.

FIG. 13 is a configuration diagram of the high-frequency treatment device according to the fourth reference example.

FIG. 14 is a flow chart which illustrates the operation of the high-frequency treatment device according to the fourth reference example.

FIG. 15 is a configuration diagram of the high-frequency treatment device according to the fourth reference example.

FIG. 16 is a configuration diagram of the high-frequency treatment device according to the first embodiment of the present invention.

FIG. 17 is a flow chart which illustrates the operation of the high-frequency treatment device according to the first embodiment of the present invention.

FIG. 18 is a chart which illustrates the relation between a current-carrying time limit and a time interval.

### DETAILED DESCRIPTION OF THE INVENTION

Hereinafter, an embodiment of this invention will be concretely described with reference to the drawings. In each of the drawings, same numerical symbols are used on the same components or corresponding components.

### Reference Example 1

The first reference example refers to the high-frequency treatment device utilizing the current-carrying region change means which changes the current-carrying region on the skin between the pair of terminal means.

Hereinafter, a contact between the skin and the terminal denotes both (A) an actual contact between the skin and the terminal and (B) an adjacent state wherein the skin and the terminal are close enough to apply the current.

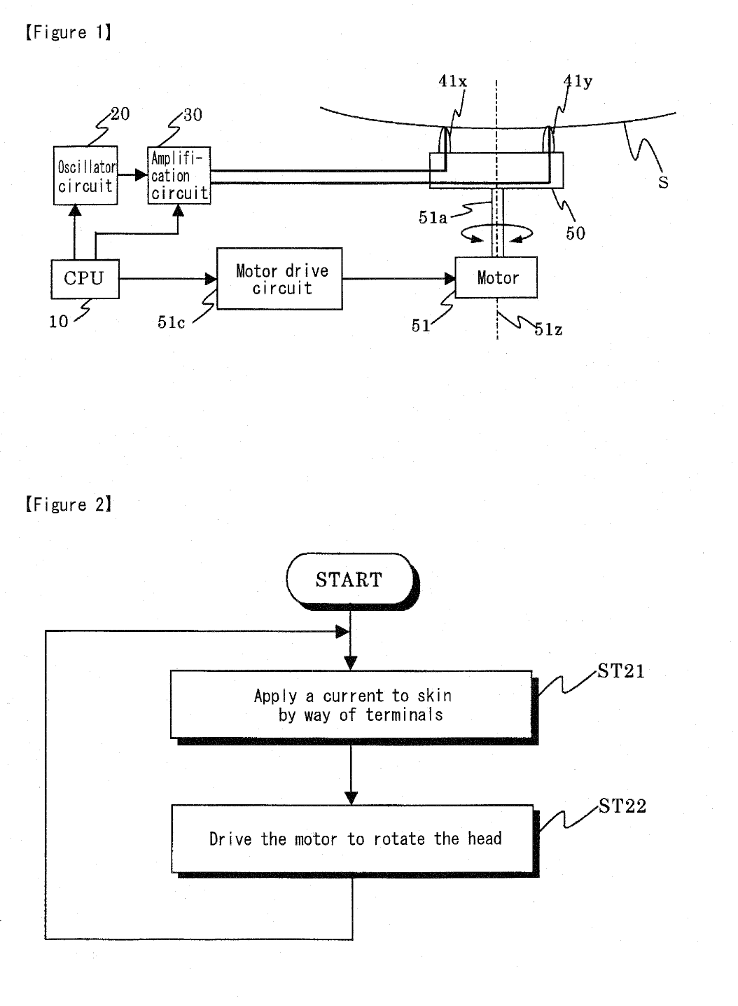

FIG. 1 is a configuration diagram of the high-frequency treatment device according to the first reference example.

In FIG. 1, a CPU (control means) which controls the high-frequency treatment device is denoted by 10, an oscillator circuit (signal generation means) which oscillates the high-frequency signal is denoted by 20, an amplification circuit (signal generation means) which amplifies the high-frequency signal is denoted by 30, terminals (terminal means) of the amplification circuit are denoted by 41x and 41y, and a head (terminal means) which holds the terminals 41x and 41y is denoted by 50.

Further, in FIG. 1, a motor (power transmission means) is denoted by 51, a junction part (power transmission means) is denoted by 51a, a motor drive circuit (power transmission means) which activates the motor 51 is denoted by 51c, a hypothetically illustrated rotating shaft of the motor 51 is denoted by 51z, and skin of the face, hands, feet or the like is denoted by S.

The head 50 is connected to the motor 51 by way of the junction part 51a. When the CPU 10 drives the motor 51 by means of the motor drive circuit 51c, the power of the motor 51 is transmitted to the head 50 by way of the junction part 51a so that the head 50 rotates around the rotating shaft 51z.

The terminals 41x and 41y placed on the head 50 contacts the skin S so that the high-frequency signal is applied to the skin S.

In FIG. 1, the current-carrying region change means is comprised of the junction part 51a, the CPU 10 and the motor drive circuit 51c.

Next, the operation will be explained.

FIG. 2 is a flow chart which illustrates the operation of the high-frequency treatment device according to the first reference example.

As shown in FIG. 2, first the CPU 10 according to FIG. 1 controls the oscillator circuit 20 and the amplification circuit 30 so that the high-frequency signal generated by the oscillator circuit 20 is amplified by the amplification circuit 30. Then the high-frequency signal from the amplification circuit 30 is applied to the skin S byway of the terminals 41x and 41y which are in contact with the skin S (Step ST21).

When the high-frequency signal is applied to the skin S, the CPU 10 drives the motor 51 by means of the motor drive circuit 51c so that the head 50 rotates around the rotating shaft 51z (Step ST22). When the head 50 rotates, the terminals 41x and 41y placed on the head 50 rotate around the rotating shaft 51z accordingly, so that the positions of the terminals 41x and 41y on the skin S can be changed.

Thereafter, the CPU 10 repeatedly carries out the steps ST21 and ST22. Therefore, the positions of the terminals 41x and 41y on the skin S continue being changed while the high-frequency signal is being applied. Accordingly, the current-carrying region between the terminals 41x and 41y continues being changed.

As described above, the CPU 10 repeatedly carries out the two steps i.e. Step ST21 wherein the high-frequency signal is applied to the skin S by way of the terminals 41x and 41y and Step ST22 wherein the terminals 41x and 41y rotate around the rotating shaft 51z. Therefore, the positions of the terminals 41x and 41y on the skin S continue being changed without staying in the same positions so that the current-carrying region continues being changed, which allows one to inhibit the excessive high-frequency signal application to the skin S, resulting in the prevention of burns or other accidents from occurring.

It is possible to control the high-frequency treatment device shown in FIG. 1 by utilizing a timer as described below.

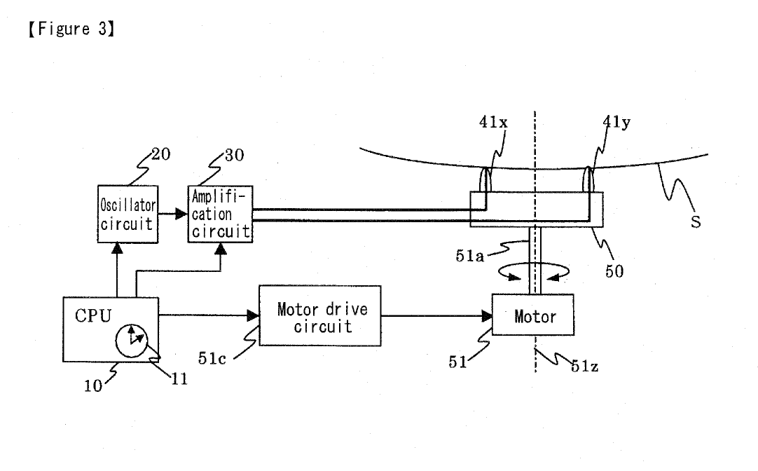

FIG. 3 is a configuration diagram of the high-frequency treatment device according to the first reference example.

In FIG. 3, a timer (timing measurement means) which measures high-frequency signal applying amount of time t1 is denoted by 11.

In FIG. 3, the current-carrying region change means is comprised of the motor 51, the junction part 51a, the timer 11, the CPU 10 and the motor drive circuit 51c.

Next, the operation will be explained.

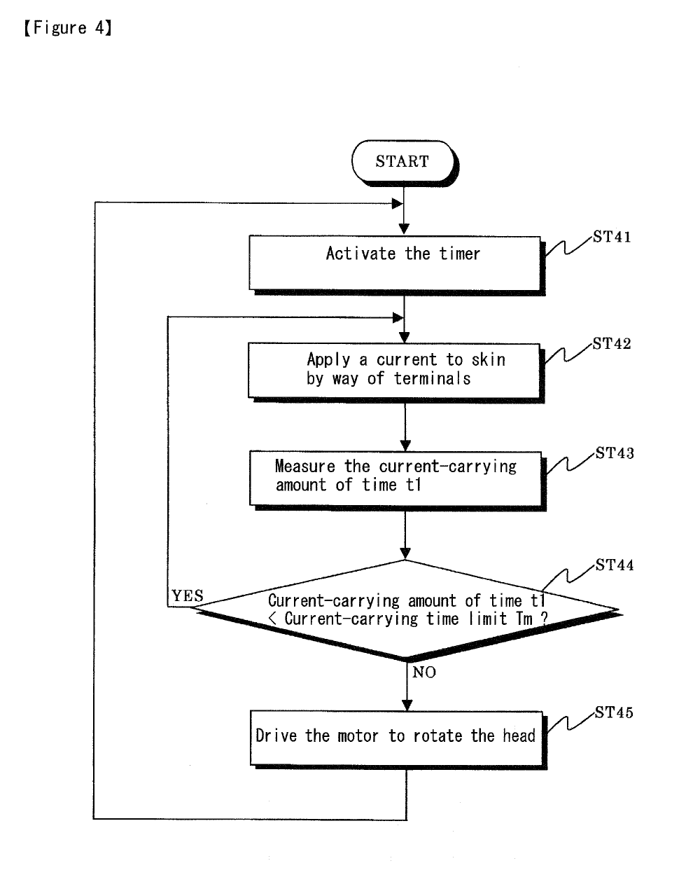

FIG. 4 is a flow chart which illustrates the operation of the high-frequency treatment device according to the first reference example.

As shown in FIG. 4, first the CPU 10 according to FIG. 3 activates the timer 11 (Step ST41) so that the high-frequency signal from the amplification circuit 30 is applied to the skin S by way of the terminals 41x and 41y (Step ST42).

Subsequently, the CPU 10 measures the current-carrying amount of time t1 (Step ST43) and determines if the current-carrying amount of time t1 reaches the current-carrying time limit Tm (Step ST44). Herein, the current-carrying time limit is defined as a specified amount of time which is considered to be at risk of danger of causing burns or other accidents if the skin S remains in the high-frequency signal application.

When the current-carrying amount of time t1 comes short of the current-carrying time limit Tm (“Yes” in Step ST44), the CPU 10 repeatedly carries out the series of processes i.e. the high-frequency signal application, the measurement of the current-carrying amount of time t1 and the comparative judgment between the current-carrying amount of time t1 and the current-carrying time limit Tm (“Yes” in Steps ST42 to ST44).

Then, when the current-carrying amount of time t1 reaches the current-carrying time limit Tm (“No” in Step ST44), the CPU 10 activates the motor 51 by means of the motor drive circuit 51c so that the head 50 rotates around the rotating shaft 51z (Step ST45) since the continued application of the high-frequency signal to the skin S carries the danger of causing burns or other accidents. When the head 50 rotates, the terminals 41x and 41y placed on the head 50 also rotate around the rotating shaft 51z accordingly so that the positions of the terminals 41x and 41y on the skin S can be changed.

Thereafter, the CPU 10 repeatedly carries out the steps ST41 to ST45. Therefore, while the high-frequency signal is being applied, the positions of the terminals 41x and 41y on the skin S can be moved every time the current-carrying amount of time t1 reaches the current-carrying time limit Tm. Accordingly, the current-carrying region between the terminals 41x and 41y can be changed.

To summarize, the CPU 10 carries out the steps as follows: Step ST42 wherein the high-frequency signal is applied to the skin S by way of the terminals 41x and 41y; Step ST43 wherein the current-carrying amount of time t1 is measured by means of the timer 11 activated in Step ST41 every time the positions of the terminals 41x and 41y on the skin S are changed; Step ST44 which determines if the current-carrying amount of time t1 reaches the current-carrying time limit Tm; and Step ST45 wherein the terminals 41x and 41y rotate around the rotating shaft 51z when the current-carrying amount of time t1 reaches the current-carrying time limit Tm i.e. “No” in Step ST44. As the current-carrying region can be changed every time the current-carrying amount of time t1 reaches the current-carrying time limit Tm, it becomes possible to inhibit the excessive high-frequency signal application to the skin S, resulting in the prevention of burns or other accidents.

A variety of embodiments can be employed in respect of the head 50.

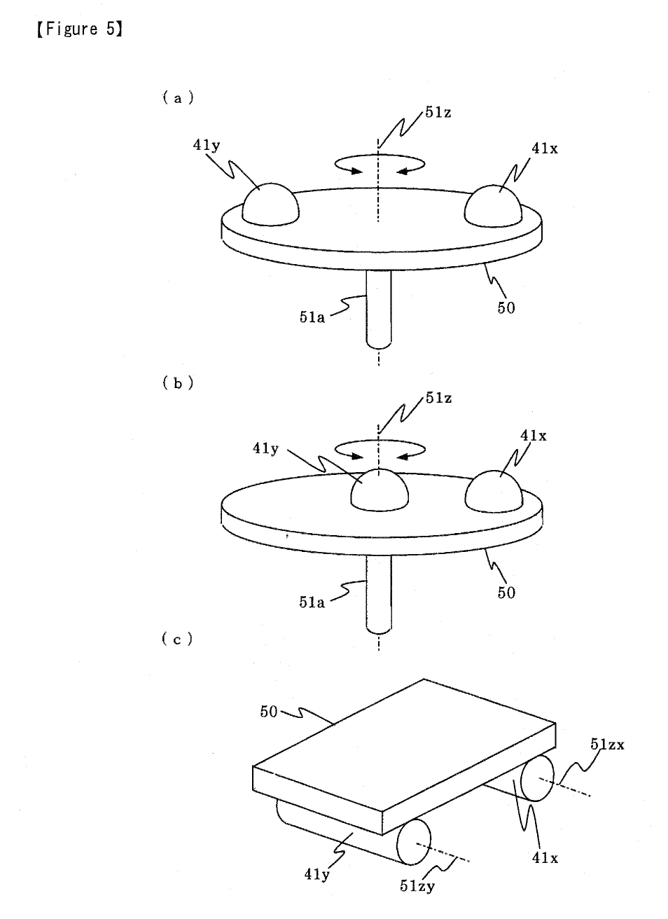

For example, as shown in FIG. 5(a), it is possible to rotatably place at least one of the terminals 41x and 41y on the head 50 centering around the axis of the rotating shaft 51z which is in a direction that penetrates the skin S, so that the motor 51 rotates the head 50 around the rotating shaft axis 51z by way of the junction part 51a. In this way, the location of terminals 41x and 41y in a simple embodiment can be achieved.

Further, as shown in FIG. 5(b), it is possible to place one of the terminals 41x and 41y on the head 50 according to FIG. 5(a) to be on-axis of the rotating shaft 51z which is in a direction that penetrates the skin S, so that the motor 51 rotates the head 50 around the axis of the rotating shaft 51z by way of the junction part 51a. In this way also, the location of terminals 41x and 41y in a simple embodiment can be achieved.

In the case of FIG. 5(b), the position of one of the terminals 41x and 41y placed on the rotating shaft 51z is not changed on the skin S, and the position of only the other one of the terminals 41x and 41y is changed. That means, although the positions of both of the terminals 41x and 41y on the skin S are changed in FIG. 1 or FIG. 3, it is possible to change the current-carrying region by changing the position of one of the terminals 41x and 41y on the skin S as shown in FIG. 5(b).

Furthermore, as shown in FIG. 5(c), it is possible to form at least one of the terminals 41x and 41y as a wheel-shaped rotating body so that the head 50 is equipped with the terminals 41x and 41y which rotate on the skin S. In this case, one or both of the terminals 41x and 41y rotates around the rotating shafts 51zx and 51zy based on the power of the motor 51 so that the terminals 41x and 41y can be moved on the skin S without difficulty.

As described above, the high-frequency treatment device according to the first reference example is comprised of the oscillator circuit 20 and the amplification circuit 30 which generate the high-frequency signal, and the head 50 having the terminals 41x and 41y which apply the high-frequency signal generated by the oscillator circuit 20 and the amplification circuit 30 to the skin S. As it is further comprised of the current-carrying region change means which changes the current-carrying region between the terminals 41x and 41y on the skin S while the high-frequency signal is being applied, it becomes possible to change the current-carrying region while the high-frequency signal is being applied so that the excessive current application to the skin S can be inhibited, resulting in the prevention of burns or other accidents.

Moreover, in the first reference example, the current-carrying region change means is comprised of the motor 51 which changes the positions of the terminals 41x and 41y on the skin S by way of the junction part 51a when it is driven by the motor drive circuit 51c, and the CPU 10 which drives the motor 51 by means of the motor drive circuit 51c while the high-frequency signal is being applied. Therefore, the positions of the terminals 41x and 41y continue being changed on the skin S so as to inhibit the excessive current application to the skin S, resulting in the prevention of burns or other accidents.

Further referring to the first reference example, the current-carrying region change means is further comprised of the timer 11 which measures the current-carrying amount of time t1. When the terminals 41x and 41y are moved, the CPU 10 measures the current-carrying amount of time t1 by means of the timer 11, and when the current-carrying amount of time t1 reaches the current-carrying time limit Tm, the motor 51 is driven by means of the motor drive circuit 51c. Accordingly, the current-carrying region can be changed every time the current-carrying amount of time t1 reaches the current-carrying time limit Tm so that the excessive current application to the skin S can be inhibited, resulting in the prevention of burns or other accidents.

Further referring to the first reference example, at least one of the terminals 41x and 41y is rotatably placed centering around the rotating shaft 51z which is in a direction that penetrates the skin S, and the motor 51 rotates at least one of the terminals 41x and 41y around the rotating shaft 51z when it is driven by the motor drive circuit 51c. Therefore, the simple composition of the head 50 can be achieved.

Further referring to the first reference example, as the other one of the terminals 41x and 41y is placed on the rotating shaft 51z, the simple composition of the head 50 can be achieved.

Still further referring to the first reference example, since at least one of the terminals 41x and 41y is formed as a wheel-shaped rotating body and the motor 51 rotates at least one of the terminals 41x and 41y when it is driven by means of the motor drive circuit 51c, the head 50 can be moved on the skin S without difficulty.

### Reference Example 2

Following the first reference example, the second reference example also refers to the high-frequency treatment device provided with the current-carrying region change means.

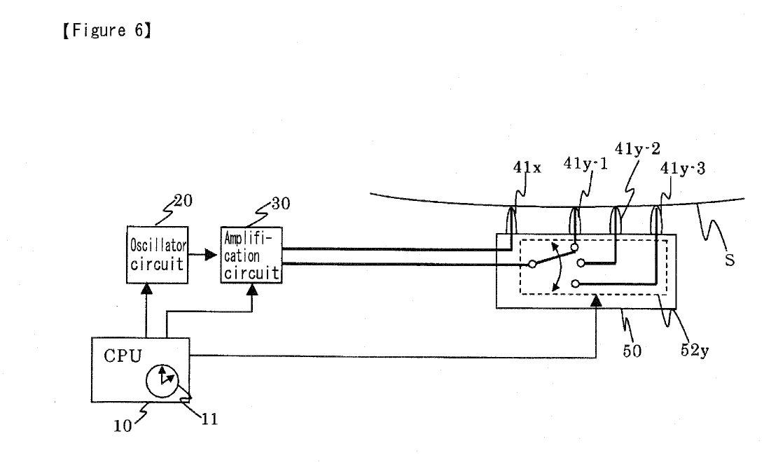

FIG. 6 is a configuration diagram of the high-frequency treatment device according to the second reference example.

In FIG. 6, three switching terminals (switching terminal means) which are electrically connectable with the amplification circuit 30 are denoted by 41y-1, 41y-2 and 41y-3, and a switch (switch means) which electrically connects to the amplification circuit 30 by choosing one from the switching terminals 41y-1 to 41y-3 is denoted by 52y.

In FIG. 6, the current-carrying region change means is comprised of the switch 52y, the timer 11 and the CPU 10.

Next, the operation will be explained.

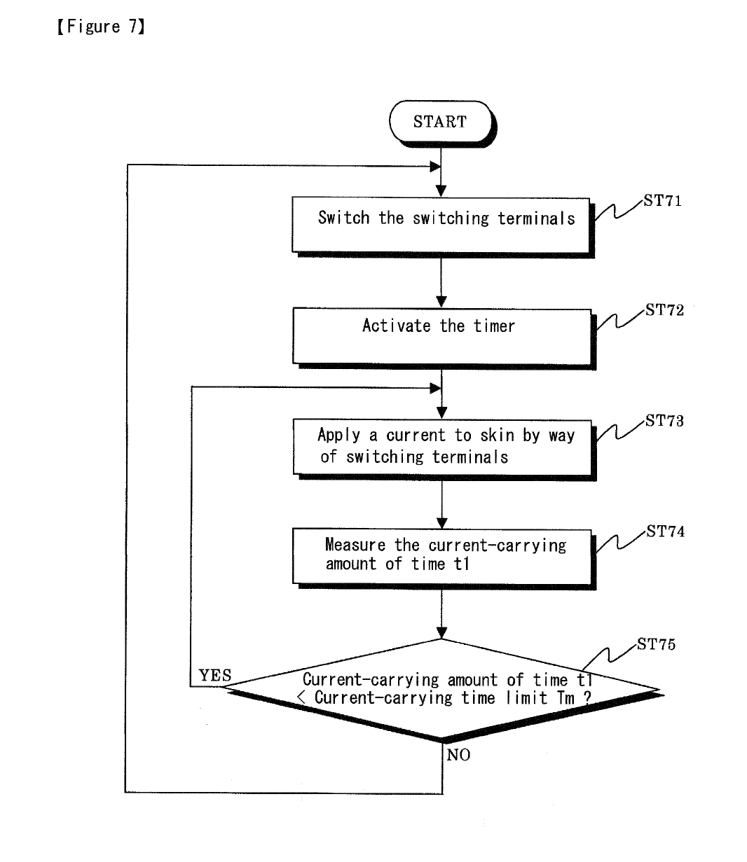

FIG. 7 is a flow chart which illustrates the operation of the high-frequency treatment device according to the second reference example.

Here, the high-frequency treatment device is used by a user pressing the head 50 against the skin S so that the terminal 41x and switching terminals 41y-1 to 41y-3 are not moved on the skin S.

In FIG. 7, first the CPU 10 according to FIG. 6 activates the switch 52y so that the switching terminal 41y-1 is switched out of the switching terminals 41y-1 to 41y-3 and the switching terminal 41y-1 is electrically connected to the amplification circuit 30 (Step ST71). Then, the CPU 10 activates the timer 11 (Step ST72) so that the high-frequency signal is applied to the skin S by way of the terminal 41x and the switching terminal 41y-1 (Step ST73).

Subsequently, the CPU 10 measures the current-carrying amount of time t1 by means of the timer 11 (Step ST74) and determines if the current-carrying amount of time t1 reaches the current-carrying time limit Tm (Step ST75).

When the current-carrying amount of time t1 comes short of the current-carrying time limit Tm (“Yes” in Step ST75), the CPU 10 repeatedly carries out the series of process i.e. the high-frequency signal application, the measurement of the current-carrying amount of time t1 and the comparative judgment between the current-carrying amount of time t1 and the current-carrying time limit Tm (“Yes” in Steps ST73 to ST75).

Then, when the current-carrying amount of time t1 reaches the current-carrying time limit Tm (“No” in Step ST75), the CPU 10 activates the switch 52y so that the switching terminal 41y-2 is switched out of the remaining switching terminals 41y-2 and 41y-3 and the switching terminal 41y-2 is electrically connected to the amplification circuit 30 (Step ST71) since further high-frequency signal application to the skin S carries the danger of causing burns or other accidents.

Thereafter, the CPU 10 activates the timer 11 again (Step ST72) so that the high-frequency signal is applied to the skin S by way of the terminal 41x and the switching terminal 41y-2 (Step ST73). Then the CPU 10 measures the current-carrying amount of time t1 by means of the timer 11 (Step ST74). Until the current-carrying amount of time t1 reaches the current-carrying time limit Tm (“No” in Steps ST74 and ST75), it repeatedly carries out the series of processes i.e. the high-frequency signal application, the measurement of the current-carrying amount of time t1 and the comparative judgment between the current-carrying amount of time t1 and the current-carrying time limit Tm (“Yes” in Steps ST73 to ST75).

After the current is applied by way of the terminal 41x and the switching terminal 41y-2 for the current-carrying time limit Tm (“No” in Step ST75), the CPU 10 activates the switch 52y so that the switching terminal 41y-3 is switched out of the remaining switching terminals 41y-3 and 41y-1 (Step ST71) and repeatedly carries out the same operations described above (Steps ST72 to ST75).

To summarize, the CPU 10 carries out the steps as follows: Step ST73 wherein the high-frequency signal is applied to the skin S by way of the terminal 41x and one of the switching terminals 41y-1 to 41y-3; Step ST74 wherein the current-carrying amount of time t1 is measured by means of the timer 11 activated in Step ST72 every time the switching terminals 41y-1 to 41y-3 is switched; Step ST75 which determines if the current-carrying amount of time t1 reaches the current-carrying time limit Tm; and Step ST71 wherein the switch 52x is activated so that one of the remaining switching terminals 41y-1 to 41y-3 is switched and the high-frequency signal from the amplification circuit 30 is applied when the current-carrying amount of time t1 reaches the current-carrying time limit Tm i.e. “No” in Step ST75. As the current-carrying region can be changed every time the current-carrying amount of time t1 reaches the current-carrying time limit Tm, it becomes possible to inhibit the excessive current application to the skin S, resulting in the prevention of burns or other accidents.

In respect of the switching terminals 41y-1 to 41y-3, it is possible to switch in order of 41y-1, 41y-2, 41y-3, 41y-1, 41y-2, 41y-3, (and repeat) in a cyclical way, or it is also possible to randomly switch one of the switching terminals 41y-1 to 41y-3.

In addition, the number of the switching terminals is not limited to three. With more than two switching terminals, the second reference example can be achieved.

Further, although it is not shown in FIG. 6, it is possible to set a single terminal on the terminal 41y side concurrently setting two or more switching terminals on the 41x side so that the two or more switching terminals can be switched when the CPU 10 activates the switch which is set correspondingly.

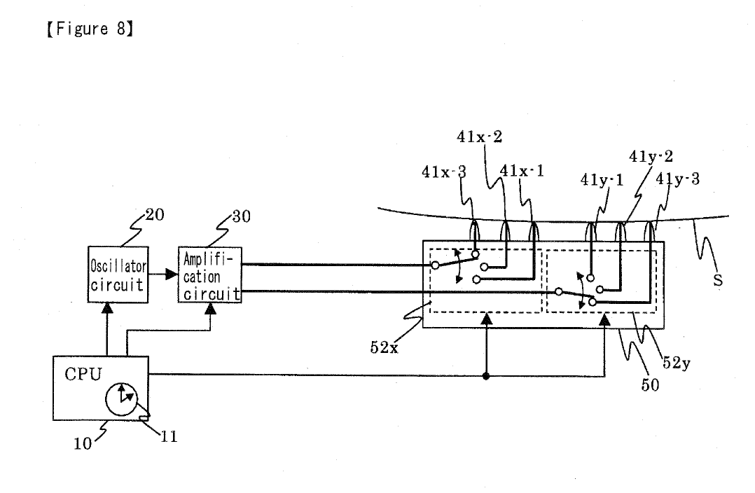

FIG. 8 shows another option.

FIG. 8 is a configuration diagram of the high-frequency treatment device according to the second reference example.

In FIG. 8, switching terminals (switching terminal means) which electrically connect to the amplification circuit 30 are denoted by 41x-1 to 41x-3. A switch (switch means) which switches the switching terminals 41x-1 to 41x-3 is denoted by 52x.

In FIG. 8, the current-carrying region change means is comprised of the switches 52x and 52y, the timer 11 and the CPU 10.

Here, the head 50 is provided with not only the set of the switching terminals 41y-1 to 41y-3 and the switch 52y but also the set of the switching terminals 41x-1 to 41x-3 and the switch 52x. When the CPU 10 activates the switches 52x and 52y, the switching terminals 41x-1 to 41x-3 and the switching terminals 41y-1 to 41y-3 are switched respectively.

Both the terminal 41x side and the terminal 41y side is provided with two or more switching terminals i.e. 41x-1 to 41x-3 or 41y-1 to 41y-3. Accordingly, comparing to the case of FIG. 6, the combinations of the switching terminals 41x-1 to 41x-3 and the switching terminals 41y-1 to 41y-3 can be varied so that the current-carrying region which is capable of being changed thereon can be widened, leading to prevent burns or other accidents from occurring more effectively. Herein, not only a concurrent switching of both the terminal 41x side and the terminal 41y side is possible but also an alternate switching or a random switching is possible.

Furthermore, the switches 52x and 52y in FIG. 6 or FIG. 8 are not necessarily placed on the head 50. They can be placed on other components.

As described above, the high-frequency treatment device according to the second reference example is provided with some means as follows: the terminal 41y is comprised of the switching terminals 41y-1 to 41y-3 which are independent of each other and apply the high-frequency signal generated by the oscillator circuit 20 and the amplification circuit 30 to the skin S; and the current-carrying region change means comprised of the switch 52y which switches the switching terminals 41y-1 to 41y-3 which apply the high-frequency signal when it is activated, the timer 11 which measures the high-frequency signal applying amount of time t1, and the CPU 10 which measures the current-carrying amount of time t1 by means of the timer 11 when the switching terminals 41y-1 to 41y-3 is switched so as to activate the switch 52y when the current-carrying amount of time t1 reaches the current-carrying time limit Tm. Consequently, the current-carrying region is changed every time the current-carrying amount of time t1 reaches the current-carrying time limit Tm so that the excessive current application to the skin S can be inhibited, resulting in the prevention of burns or other accidents.

Further, the high-frequency treatment device according to the second reference example is provided with some means as follows: the terminals 41x and 41y are comprised of the switching terminals 41x-1 to 41x-3 and 41y-1 to 41y-3 which are independent of each other and apply the high-frequency signal generated by the oscillator circuit 20 and the amplification circuit 30 to the skin S; and the current-carrying region change means, comprised of the switches 52x and 52y which are set corresponding to the terminals 41x and 41y and switches the switching terminals 41x-1 to 41x-3 and 41y-1 to 41y-3 which apply the high-frequency signal when it is activated, the timer 11 which measures the high-frequency signal applying amount of time t1, and the CPU 10 which measures the current-carrying amount of time t1 by means of the timer 11 when the switching terminals 41x-1 to 41x-3 and 41y-1 to 41y-3 are switched so as to activate the switches 52x and 52y when the current-carrying amount of time t1 reaches the current-carrying time limit Tm. Consequently, comparing to the case of FIG. 6, the combinations of the switching terminals 41x-1 to 41x-3 and 41y-1 to 41y-3 can be varied so that the current-carrying region which is capable of being changed thereon can be widened, resulting in the prevention of burns or other accidents from occurring more effectively.

### Reference Example 3

The third reference example refers to the high-frequency treatment device utilizing the signal application control means which determines whether the skin region is suited for the high-frequency signal application so as to control the high-frequency signal application based on the result thereof.

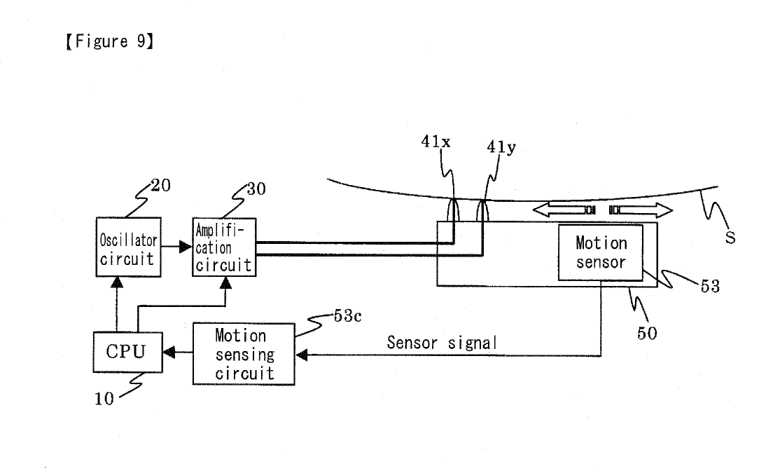

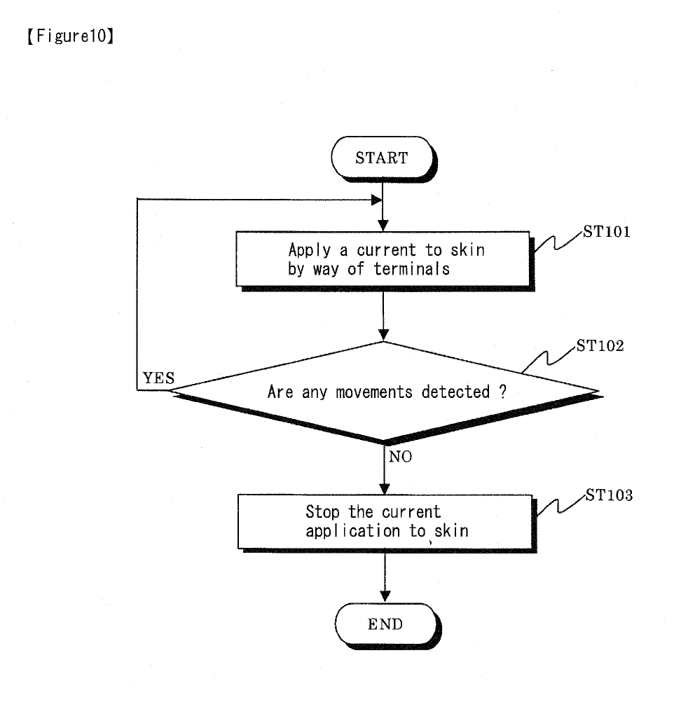

FIG. 9 is a configuration diagram of the high-frequency treatment device according to the third reference example.

In FIG. 9, a motion sensor (motion sensing means) which outputs a sensor signal by way of self-motion-sensing is denoted by 53. A motion sensing circuit (motion sensing means) which senses movements by way of the sensor signal output from the motion sensor 53 is denoted by 53c.

The motion sensor 53 employs a motion sensing method which emits light to the skin S to receive its reflected light so that the self-motion-sensing can be achieved, which is the same method as an optical mouse. As the motion sensor is placed on the head 50 together with the terminals 41x and 41y, sensing of movements of the terminals 41x and 41y onto the skin S can be achieved.

In FIG. 9, the signal application control means is comprised of the motion sensor 53, the motion sensing circuit 53c and the CPU 10.

Next, the operation will be explained.

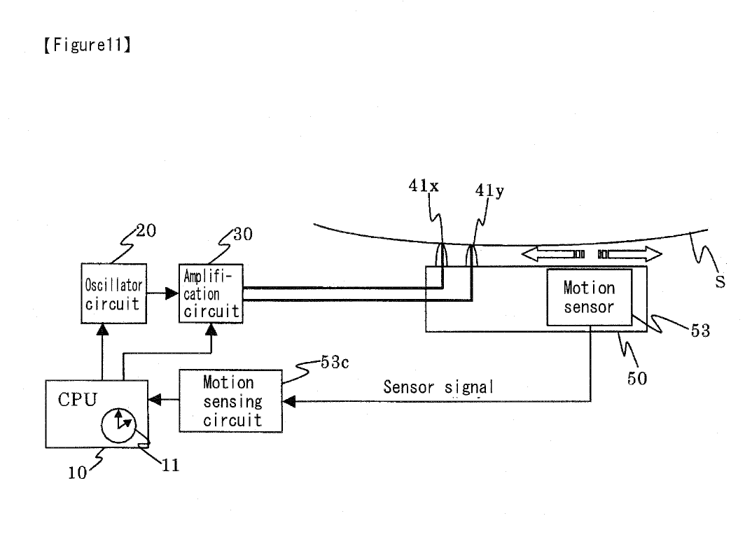

FIG. 10 is a flow chart which illustrates the operation of the high-frequency treatment device according to the third reference example. Here, the high-frequency treatment device is used by a user moving the same so that the terminals 41x and 41y are moved on the skin S.

In FIG. 10, first a high-frequency signal is applied to the skin S by way of the terminals 41x and 41y according to FIG. 6 (Step ST101). Then, the motion sensor 53 senses movements of the terminals 41x and 41y so as to output a sensor signal, which is eventually transferred to the motion sensing circuit 53c. The motion sensing circuit 53c senses movements by way of the sensor signal which is transmitted to the CPU 10 (Step ST102).

While any movements are being sensed (“Yes” in Step ST102), the terminals 41x and 41y are logically being moved on the skin S. Accordingly, the CPU 10 determines no danger of excessive current application to the skin S and controls the oscillator circuit 20 and the amplification circuit 30 so as to continue the high-frequency signal application (Step ST101).

On the other hand, when no movements have been sensed (“No” in Step ST102), the terminals 41x and 41y are not logically being moved on the skin S. Accordingly, the CPU 10 determines a danger of excessive current application to the skin S and controls the oscillator circuit 20 and the amplification circuit 30 so as to stop the high-frequency signal application (Step ST103).

To summarize, the CPU 10 carries out the steps as follows: Step ST101 wherein the high-frequency signal is applied to the skin S by way of the terminals 41x and 41y; Step ST102 wherein movements are sensed by means of the motion sensor 53 and the motion sensing circuit 53c; and Step ST103 wherein the current application to the skin S is stopped when no movements have been sensed i.e. when “No” in Step ST102. As no high-frequency signal is applied unless the terminals 41x and 41y are being moved, it becomes possible to inhibit the excessive high-frequency signal application to the skin S, resulting in the prevention of burns or other accidents.

Below is another option with the timer 11.

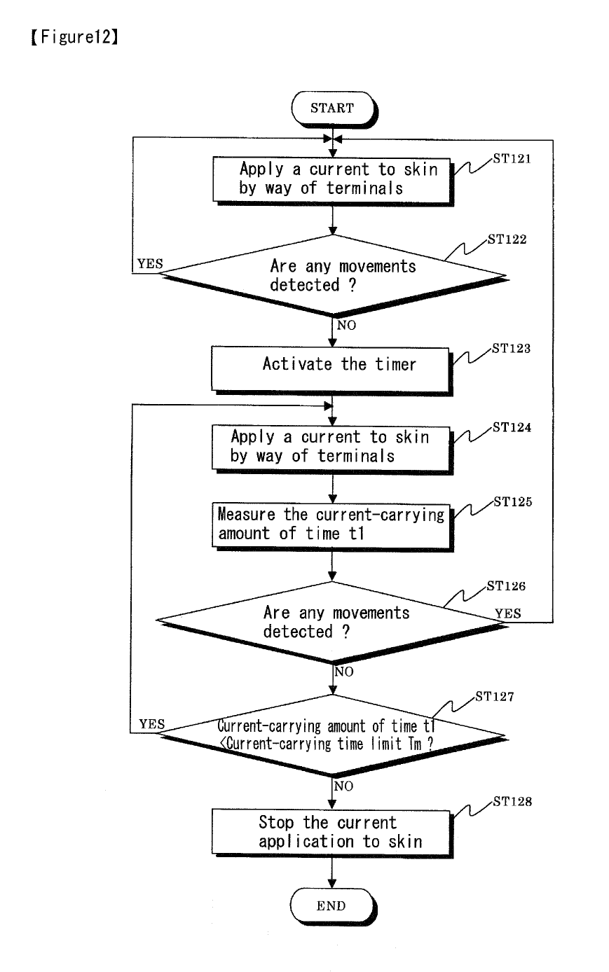

FIG. 11 is a configuration diagram of the high-frequency treatment device according to the third reference example.

In FIG. 11, the signal application control means is comprised of the motion sensor 53, the motion sensing circuit 53c, the timer 11 and the CPU 10.

Next, the operation will be explained.

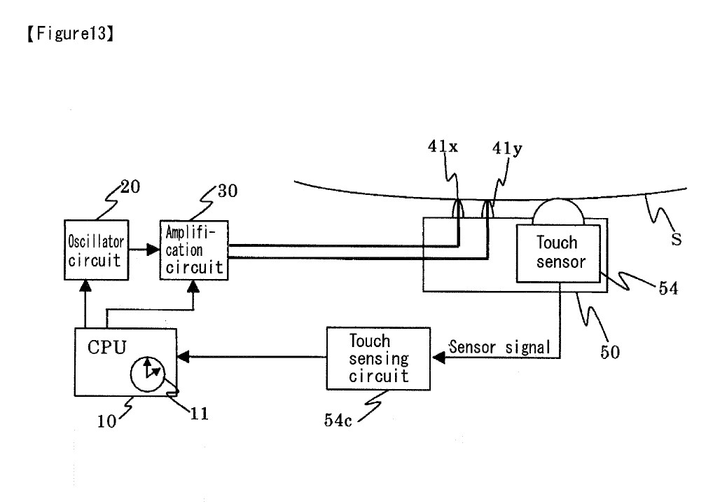

FIG. 12 is a flow chart which illustrates the operation of the high-frequency treatment device according to the third reference example.

In FIG. 12, even if movements have become undetectable while the high-frequency signal is being applied (“No” in Steps ST121 and ST122), the CPU 10 according to FIG. 11 does not stop the current application immediately, instead the timer 11 is activated and the current application is continued (Steps ST123 and ST124). Then the current-carrying amount of time t1 from the point that movements have become undetectable is measured by means of the timer 11 (Step ST125).

When any movement has been sensed while t1 is being measured (“Yes” in Step ST126), the terminals 41x and 41y are logically being moved on the skin S. Accordingly, the CPU 10 determines that the danger of excessive current application to the skin S has been resolved and goes back to the series of Steps ST121 and ST122.

On the other hand, when no movement has been sensed (“No” in Steps ST126), the CPU 10 determines whether the current-carrying amount of time t1 reaches the current-carrying time limit Tm (Step ST127). When the current-carrying amount of time t1 comes short of the current-carrying time limit Tm (“Yes” in Step ST127), the CPU 10 repeatedly carries out the series of process i.e. the high-frequency signal application, the measurement of the current-carrying amount of time t1 and the comparative judgment between the current-carrying amount of time t1 and the current-carrying time limit Tm (“Yes” in Steps ST124 to ST127).

Thereafter, the high-frequency signal application and the measurement of the current-carrying amount of time t1 are repeatedly carried out (Steps ST124 and ST125). When no movement has been sensed (“No” in Step ST126) and eventually the current-carrying amount of time t1 reaches the current-carrying time limit Tm (“No” in Step ST127), the CPU 10 determines that further high-frequency signal application to the skin S carries the danger of causing burns or other accidents and stops the current application forcibly (Step ST128).

To summarize, the CPU 10 carries out the steps as follows: Step ST124 wherein the high-frequency signal is being applied by way of the terminals 41x and 41y when no movements of the terminals 41x and 41y have been sensed while the high-frequency signal is being applied i.e. when “No” in Step ST122; Step ST125 which measures the current-carrying amount of time t1 by means of the timer 11 activated in Step ST123; Step ST127 which determines whether the current-carrying amount of time t1 reaches the current-carrying time limit Tm when no movements have been sensed in Step ST126; and Step ST128 wherein the current application is stopped forcibly when the current-carrying amount of time t1 reaches the current-carrying time limit Tm i.e. when “No” in Step ST127. Accordingly, the current application is carried out from the point at which movements have become undetectable until the current-carrying time limit Tm, and thereafter it is stopped. Therefore, the excessive current application to the skin S is inhibited so that the burns or other accidents can be prevented.

As for the movements of the terminals 41x and 41y sensed by the motion sensor 53 and the motion sensing circuit 53c, when one of the terminals 41x and 41y is moved on the skin S, the current-carrying region is logically moved so that the high-frequency signal application can be carried out. When both of the terminals 41x and 41y are kept motionless, the current-carrying region is logically kept in the same position so that the current application is stopped.

The method employed by the motion sensor 53 is not limited to the self-motion-sensing method as in an optical mouse. It is only necessary to be capable of sensing movements of the terminals 41x and 41y to the skin S. For example, it is possible to employ the following methods (1) to (3) for the motion sensing means.

(1) An acceleration-sensor-method wherein the head 50 is provided with an acceleration sensor which senses an acceleration.

(2) A gyro-sensor-method wherein the head 50 is provided with a gyro sensor which senses an angular velocity.

(3) A motor-rotation-sensor-method wherein the head 50 is provided with a motion sensing motor which senses an impetus generated by rotating. The rotating is frictionally generated when a drive part of the motion sensing motor moves on the skin S while the current is being applied.

As described above, the high-frequency treatment device according to the third reference example is comprised of the oscillator circuit 20 and the amplification circuit 30 which generate the high-frequency signal and the terminals 41x and 41y which apply the high-frequency signal generated by the oscillator circuit 20 and the amplification circuit 30. It further comprises the signal application control means which determines whether the skin region is suited for the high-frequency signal application so as to control the high-frequency signal application based on the result thereof. Accordingly, it becomes possible to control the high-frequency signal application properly corresponding to the evaluation so that the excessive current application to the skin S can be inhibited, resulting in the prevention of burns or other accidents.

In addition, the signal application control means according to the third reference example is comprised of the motion sensor 53 and the motion sensing circuit 53c which senses movements of the terminals 41x and 41y onto the skin S and the CPU 10 which stops the oscillator circuit 20 and controls the amplification circuit 30 so as to apply the high-frequency signal while any movements of the terminals 41x and 41y are being sensed by the motion sensor 53 and the motion sensing circuit 53c. As the high-frequency signal is applied when the terminals 41x and 41y are not staying in the same positions on the skin S, it becomes possible to inhibit the excessive current application to the skin S, resulting in the prevention of burns or other accidents.

Further referring to the third reference example, when movements of the terminals 41x and 41y have become undetectable by the motion sensor 53 and the motion sensing circuit 53c, the CPU 10 controls the oscillator circuit 20 and the amplification circuit 30 so as to stop the high-frequency signal application. Accordingly, no high-frequency signal is applied while the terminals 41x and 41y are staying in the same positions on the skin S so that the excessive current application to the skin S can be inhibited, resulting in the prevention of burns or other accidents.

Further referring to the third reference example, the signal application control means is further comprised of the timer 11 which measures the high-frequency signal applying amount of time t1. The CPU 10 measures the current-carrying amount of time t1 by means of the timer 11 when movements of the terminals 41x and 41y have become undetectable by the motion sensor 53 and the motion sensing means 53c. Further, when the current-carrying amount of time t1 reaches the current-carrying time limit Tm, the CPU 10 controls the oscillator circuit 20 and the amplification circuit 30 so as to stop the high-frequency signal application. Accordingly, the high-frequency signal application is carried out from the point at which the terminals 41x and 41y have stayed in the same position on the skin S until the current-carrying time limit Tm, and thereafter it is stopped. Therefore, the excessive current application to the skin S is inhibited so that burns or other accidents can be prevented.

### Reference Example 4

Following the third reference example, the fourth reference example also refers to the high-frequency treatment device provided with the signal application control means.

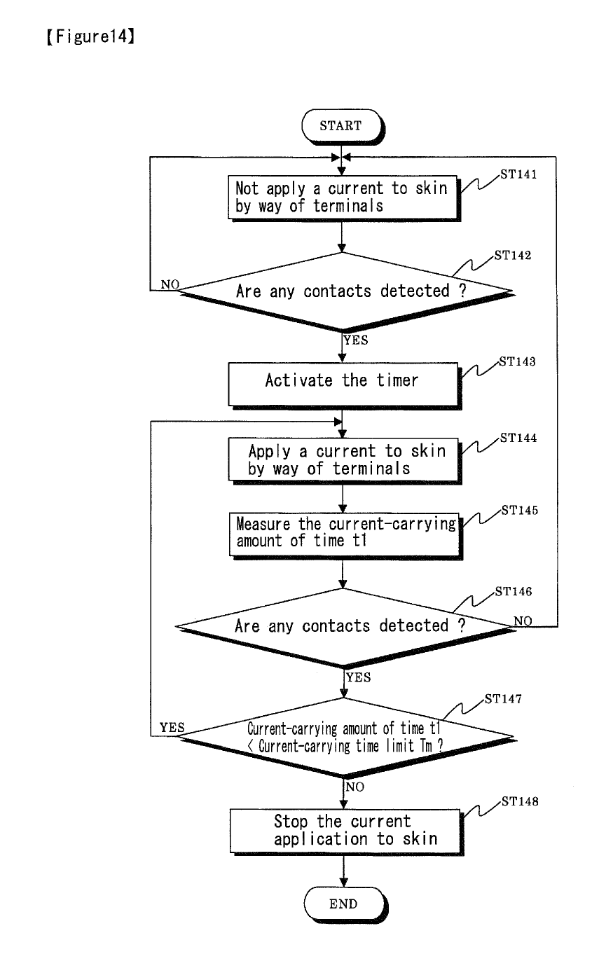

FIG. 13 is a configuration diagram of the high-frequency treatment device according to the fourth reference example.

In FIG. 13, a touch sensor (contact sensing means) which senses a contact between itself and the skin S and outputs a sensor signal is denoted by 54, and a touch sensing circuit (contact sensing means) which senses a contact by way of the sensor signal output from the touch sensor 54 is denoted by 54c.

The touch sensor 54 is placed on the head 50 together with the terminals 41x and 41y. When the touch sensor 54 contacts the skin S, the terminals 41x and 41y also contact the skin S. Therefore, since the touch sensor 54 senses the contact between itself and the skin S, it logically senses the contact between the terminals 41x and 41y and the skin S as well.

In FIG. 13, the signal application control means is comprised of the touch sensor 54, the touch sensing circuit 54c, the timer 11 and the CPU10.

Next, the operation will be explained.

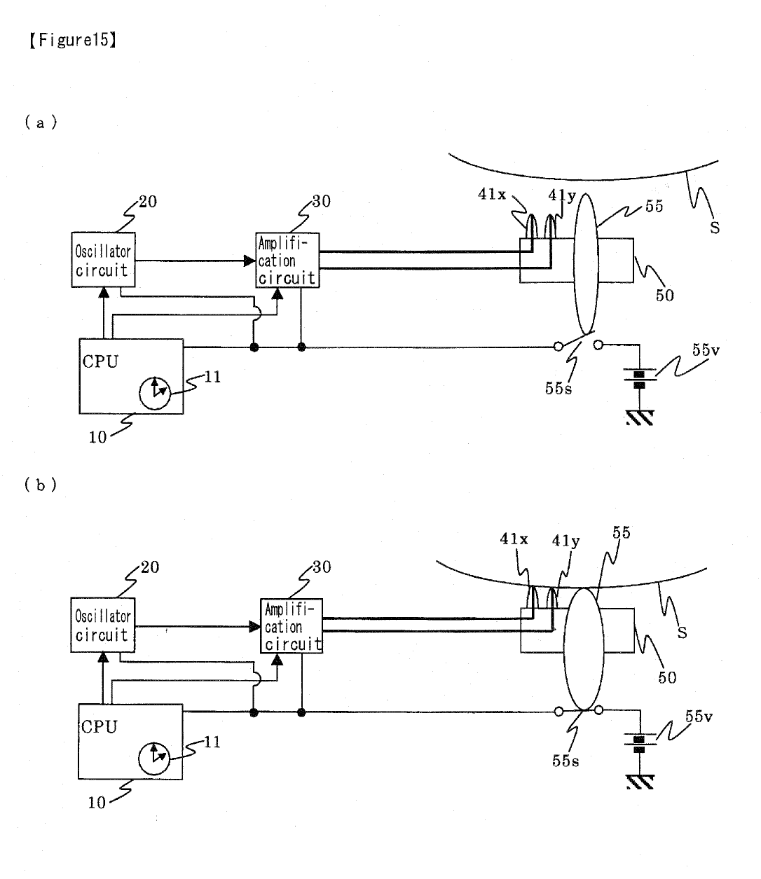

FIG. 14 is a flow chart which illustrates the operation of the high-frequency treatment device according to the fourth reference example.

In FIG. 14, the touch sensor 54 according to FIG. 13 senses the contact between the terminals 41x and 41y and the skin S so as to output the sensor signal which is transferred to the touch sensing circuit 54c. The touch sensing circuit 54c senses the contact by way of the sensor signal which is eventually transferred to the CPU 10 (Step ST142).

While no contacts have been sensed (“No” in Step ST142), the CPU 10 controls the oscillator 20 and the amplification circuit 30 so as not to apply the high-frequency signal to the skin S, and is maintained in a standby mode (Step ST141).

Then, when a contact has been sensed (“Yes” in Step ST142), the CPU 10 determines the need of the high-frequency signal application and activates the timer 11 so as to start the current application (Steps ST143 and ST144), and measures the current-carrying amount of time t1 by means of the timer 11 (Step ST145).

When contacts have stopped being sensed while t1 is being measured (“Yes” in Step ST146), the CPU 10 determines that the high-frequency signal application is no longer needed and goes back to the series of Steps ST141 and ST142.

On the other hand, when any contact has been sensed while the current is being applied (“Yes” in Steps ST144 to ST146), the CPU 10 determines whether the current-carrying amount of time t1 reaches the current-carrying time limit Tm (Step ST147). When the current-carrying amount of time t1 comes short of the current-carrying time limit Tm (“Yes” in Step ST147), the CPU 10 repeatedly carries out the series of process i.e. the high-frequency signal application, the measurement of the current-carrying amount of time t1 and the comparative judgment between the current-carrying amount of time t1 and the current-carrying time limit Tm (“Yes” in Steps ST144 to ST147).

Thereafter, the high-frequency signal application and the measurement of the current-carrying amount of time t1 are repeatedly carried out (Steps ST144 and ST145). When any contact has been sensed (“Yes” in Step ST146) and eventually the current-carrying amount of time t1 reaches the current-carrying time limit Tm (“No” in Step ST147), the CPU 10 determines that further high-frequency signal application to the skin S carries the danger of causing burns or other accidents and stops the current application forcibly (Step ST148).

After the stop of the current application (Step ST148), when the user removes the head 50 from the target human body so that the touch sensing circuit 54c communicates with the CPU 10 that contacts between the terminals 41x and 41y and the skin S is no longer sensed, the entire high-frequency treatment device is reset. Thereafter the CPU 10 carries out the process which starts from the Step ST141 again.

To summarize, the CPU 10 carries out the steps as follows: Step ST144 wherein the high-frequency signal is applied by way of the terminals 41x and 41y when contacts between the terminals 41x and 41y and the skin S have been sensed i.e. when “Yes” in Step ST142; Step ST145 which measures the current-carrying amount of time t1 by means of the timer 11 activated at Step ST143; Step ST147 which determines whether the current-carrying amount of time t1 reaches the current-carrying time limit Tm when any contacts have been sensed at Step ST146; and Step ST148 which forcibly stops the current application when the current-carrying amount of time t1 reaches the current-carrying time limit Tm i.e. when “No” in Step ST147. Accordingly, the current application is carried out from the point at which movements has been sensed until the current-carrying time limit Tm, and thereafter it is stopped. Therefore, the excessive current application to the skin S is inhibited so that the burns or other accidents can be prevented.

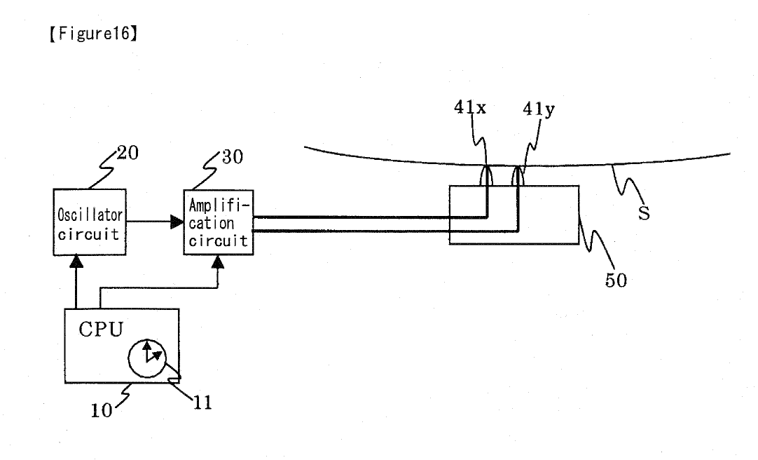

Here, sensing methods employed by the touch sensor 54 can be, for example, a method wherein changes of the electric capacity or the impedance are sensed or a method wherein pressure changes are sensed by means of a piezoelectric element. Other than those above, FIG. 15 shows another option.

FIG. 15 is a configuration diagram of the high-frequency treatment device according to the fourth reference example.

In FIG. 15, an elastic member (contact sensing means) is denoted by 55, a switch (control means) is denoted by 55s and a power source (control means) of the high-frequency treatment device is denoted by 55v.

In FIG. 15, the signal application control means is comprised of the elastic member 55, the switch 55s, the power source 55v, the timer 11 and the CPU 10.

When the elastic member 55 contacts the skin S to be pressed thereto so that the condition is changed from the one shown in FIG. 15(a) to the one shown in FIG. 15(b), the elastic member 55 switches on the switch 55s. Accordingly, the power from the power source 55v is supplied to the high-frequency treatment device. Thereafter the same operations as in FIG. 14 follow.

As described above, the signal application control means according to the fourth reference example is provided with some means as follows: the touch sensor 54 and the touch sensing circuit 54c which sense the contact between the terminals 41x and 41y and the skin S; the timer 11 which measures the high-frequency signal applying amount of time t1; and the CPU 10 which applies the high-frequency signal by controlling the oscillator circuit 20 and the amplification circuit 30 when contacts between the terminals 41x and 41y and the skin S have been sensed by the touch sensor 54 and the touch sensing circuit 54c, and measures the current-carrying amount of time t1 by means of the timer 11, and stops the high-frequency signal application by controlling the oscillator circuit 20 and the amplification circuit 30 when the current-carrying amount of time t1 reaches the current-carrying time limit Tm. Accordingly, the high-frequency signal application is kept being carried out from the point at which any contacts between the terminals 41x and 41y and the skin S have been sensed until the current-carrying time limit Tm, and thereafter it is stopped. Therefore, the excessive current application to the skin S is inhibited so that burns or other accidents can be prevented.

### Embodiment 1

Following the third and fourth reference examples, the first embodiment also refers to the high-frequency treatment device provided with the signal application control means.

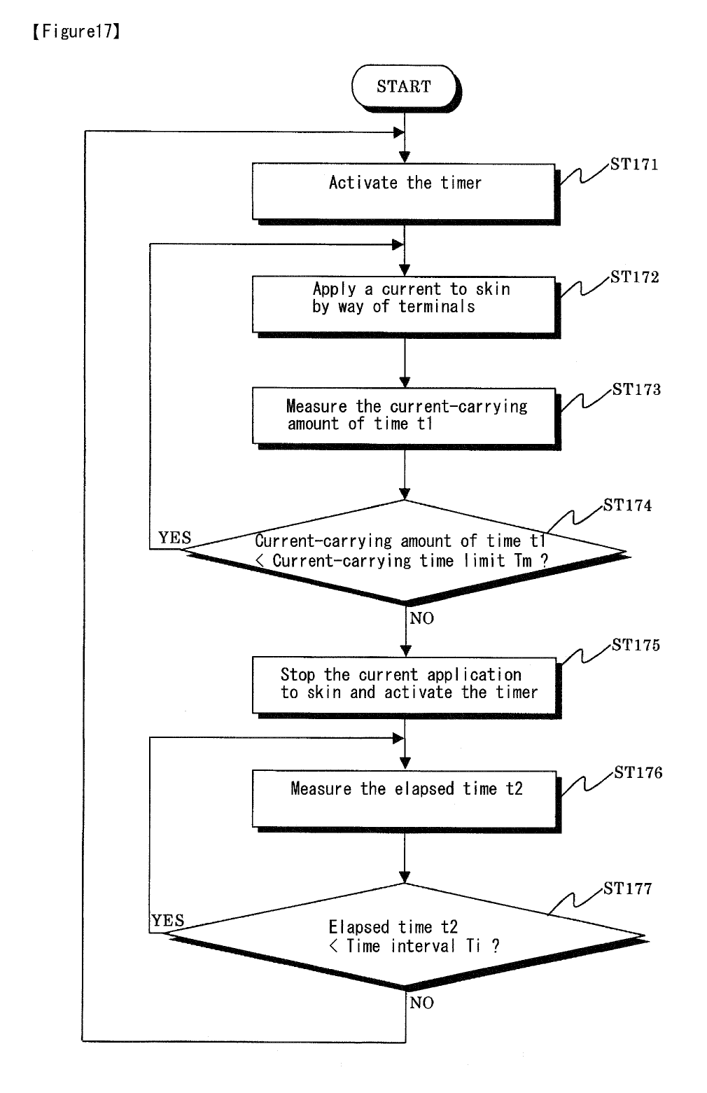

FIG. 16 is a configuration diagram of the high-frequency treatment device according to the first embodiment of the present invention.

In FIG. 16, the signal application control means is comprised of the timer 11 and the CPU 10.

Next, the operation will be explained.

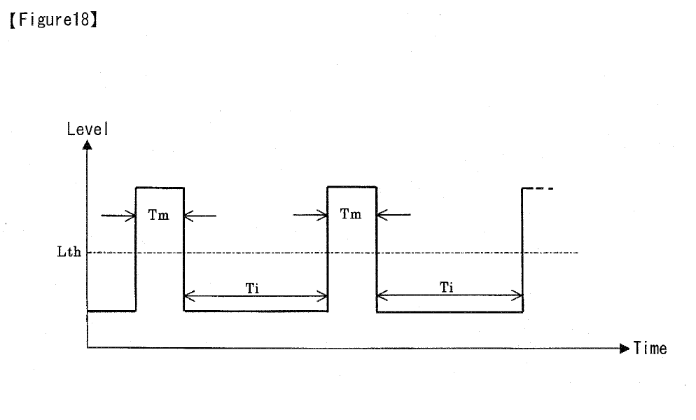

FIG. 17 is a flow chart which illustrates the operation of the high-frequency treatment device according to the first embodiment of the present invention.

In FIG. 17, for example when an operation button which is not shown in FIG. 16 is pressed by a user, the CPU 10 according to FIG. 16 determines the need of high-frequency signal application and activates the timer 11 so as to start the current application (Steps ST171 and ST172), and measures the current-carrying amount of time t1 by means of the timer 11 (Step ST173). Subsequently, the CPU 10 repeatedly carries out the series of process i.e. the high-frequency signal application, the measurement of the current-carrying amount of time t1 and the comparative judgment between the current-carrying amount of time t1 and the current-carrying time limit Tm (“Yes” in Steps ST172 to ST174).

Thereafter, the high-frequency signal application and the measurement of the current-carrying amount of time t1 are repeatedly carried out (Steps ST172 and ST173). When eventually the current-carrying amount of time t1 reaches the current-carrying time limit Tm (“No” in Step ST174), the CPU 10 determines that further high-frequency signal application to the skin S carries the danger of causing burns or other accidents and stops the current application forcibly, concurrently activating the timer 11 again (Step ST175).

Then the CPU 10 measures the elapsed time from the current application stop t2 (Step ST176) and maintains the current application in the stopped state until the elapsed time t2 reaches the time interval Ti even if the operation button is being pressed (“Yes” in Steps ST176 and ST177). Herein, as shown in FIG. 18, the time interval Ti is defined as a predetermined time period which is considered long enough to avoid the danger of causing burns or other accidents if the current is re-applied to the same region on the skin S for less time than the current-carrying time limit Tm as long as the current application is maintained in the stopped state for the time interval Ti.

After some time has elapsed and eventually the elapsed time t2 reaches the time interval Ti (“No” in Step ST177), the CPU 10 determines that the danger of excessive current application to the skin S has been resolved to re-apply the current, and re-start the current application (Step ST171).

To summarize, the CPU 10 carries out the steps as follows: Step ST175 which stops the current application and activates the timer 11 when the current-carrying amount of time t1 reaches the current-carrying time limit Tm i.e. when “No” in Step ST174 while the high-frequency signal according to Step ST172 is being applied; Step ST176 which measures the elapsed time t2 by means of the timer 11 activated at Step ST175; Step ST177 which determines whether the elapsed time t2 reaches the time interval Ti; and Step ST171 which re-starts the current application when the elapsed time t2 reaches the time interval Ti i.e. when “No” in Step ST 177. Accordingly, the time interval Ti is inserted between the current applications so that the excessive current application to the skin S can be inhibited, resulting in the prevention of burns or other accidents.

Although it is not shown in FIG. 17, another option is described below.

That is, the level of the current applied during the time interval Ti is not necessarily maintained at zero. It is possible that the CPU 10 controls the oscillator circuit 20 and the amplification circuit 30 so as to apply the high-frequency signal at a level which is considered weak enough to avoid burns or accidents i.e. a level which is below the specified threshold value Lth.

Then, after the time interval Ti has elapsed, the CPU 10 controls the oscillator circuit 20 and the amplification circuit 30 so as to re-apply the current by increasing the high-frequency signal level above the threshold value Lth. That is to say, the current application stops, as in FIG. 17, when the threshold value Lth is zero.

In addition, although the high-frequency treatment device can employ the first embodiment independently, it is possible to employ a combination of the third and fourth reference examples and the first embodiment.

As described above, the signal application control means according to the first embodiment is provided with some means as follows: the timer 11 which measures the high-frequency signal application time t1; and the CPU 10 which measures the current-carrying amount of time t1 by means of the timer 11 when the oscillator circuit 20 and the amplification circuit 30 are controlled so that the level of the high-frequency signal is increased above the threshold value Lth, and further decrease the level of the high-frequency signal below the threshold value Lth by controlling the oscillator circuit 20 and the amplification circuit 30 when the current-carrying amount of time t1 reaches the current-carrying time limit Tm. Accordingly, the high-frequency signal whose level is above the threshold value Lth is not applied so that the excessive current application to the skin S can be inhibited, resulting in the prevention of burns or other accidents.

Further referring to the first embodiment, when the level of the high-frequency signal is decreased below the threshold value Lth, the CPU 10 measures the elapsed time t2 by means of the timer 11. Further, when the elapsed time t2 reaches the specified time interval Ti, the CPU 10 controls the oscillator circuit 20 and the amplification circuit 30 so that the level of the high-frequency signal is increased above the threshold value Lth. Accordingly, the time interval Ti is inserted between the high-frequency signal applications so that the excessive current application to the skin S can be inhibited, resulting in the prevention of burns or other accidents.

According to the first through the fourth reference examples and the first embodiment, the respective controls are conducted based on the steps as follows: the current-carrying amount of time t1 or the elapsed time t2 is measured in order to perceive the current-carrying condition; and the measured time t1 and t2 are compared to the predetermined conditions such as the current-carrying time limit Tm or the time interval Ti. However, embodiments of the present invention are not limited to those above. It is possible that the CPU 10 utilizes the temperature sensor (temperature measurement means) which measures the temperature of the current-carrying region on the skin S.

Specifically, the temperature sensor as another current-carrying condition measurement means of the timer 11 measures the temperature of the current-carrying region as a current-carrying condition. The temperature as a result thereof is compared to the predetermined conditions such as the current-carrying limit temperature or the current-carrying safe temperature. Then the CPU 10 carries out the same process as in the first through the fourth reference examples and the first embodiment, that is, the operations which are carried out when the current-carrying amount of time t1 or the elapsed time t2 reach the current-carrying time limit Tm or the time interval Ti.

Herein, the current-carrying limit temperature is defined as a specified temperature which is considered to be at risk of danger of causing burns or other accidents if the skin S remains in the high-frequency signal application. The current-carrying safe temperature is defined as a specified maximum temperature which is considered safe enough to avoid burns or other accidents if the skin S remains in the high-frequency signal application.

As described above, it is possible to employ the temperature sensor instead of the timer 11 according to the first to the fourth reference examples and the first embodiment of the present invention. As the timer 11 or the temperature sensor is utilized as the current-carrying condition measurement means, it becomes possible to perceive the current-carrying condition by measuring the time or the temperature without difficulty so that the simple composition of the current-carrying condition measurement means can be achieved.

### EXPLANATION OF SYMBOLS

10 CPU (Control means)
11 Timer (Current-carrying condition measurement means, Timing measurement means)
20 Oscillator circuit (Signal generation means)
30 Amplification circuit (Signal generation means)
41x, 41y Terminal (Terminal means)
41x-1˜41x-3, 41y-1˜41y-3 Switching terminal (Switching terminal means)
50 Head (Terminal means)
51 Motor (Power transmission means)
51a Junction part (Power transmission means)
51c Motor drive circuit (Power transmission means)
51z Rotating shaft
52x, 52y Switch (Switch means)
53 Motion sensor (Motion sensing means)
53c Motion sensing circuit (Motion sensing means)
54 Touch sensor (Contact sensing means)
54c Touch sensing circuit (Contact sensing means)
55 Elastic member (Contact sensing means)
55s Switch (Control means)
55v Power source (Control means)
S Skin
t1 Current-carrying amount of time
t2 Elapsed time
Tm Current-carrying time limit
Ti Time interval
Lth Threshold value

1-19. (canceled)

20. A high-frequency treatment device, comprising:
a signal generation means for which generating a high-frequency signal,
a pair of terminal means for applying the high-frequency signal generated by said signal generation means to skin,
a motion sensing means for sensing movements of said pair of terminal means onto skin, and
a control means for which controlling said signal generation means as follows:
while movements of at least one of said pair of terminals are being sensed by said motion sensing means, the level of the high-frequency signal is increased above a specified threshold value, and
when movements of said pair of terminals have become undetectable by said motion sensing means, the level of the high-frequency signal is decreased not to be more than the specified threshold value.

21. The high-frequency treatment device according to claim 20 further comprising a current-carrying condition measurement means for measuring a high-frequency signal application condition, such that with respect to said control means:
when movements of said pair of terminals have been undetectable by said motion sensing means, the current-carrying condition is measured by said current-carrying condition measurement means, and
when said current-carrying condition reaches a specified condition, the level of the high-frequency signal is decreased not to be more than the specified threshold value.
